(19) United States
(12) Patent Application Publication
(10) Pub. No.: US 2022/0333151 A1
(43) Pub. Date: Oct. 20, 2022

(54) Plant Microbiome and Methods for Profiling Plant Microbiome

(71) Applicant: Agriculture Victoria Services PTY LTD, Bundoora, Victoria (AU)

(72) Inventors: Tongda Li, Southbank (AU); Ian Ross Tannenbaum, Bundoora (AU); Ankush Chandel, Bundoora (AU); Jatinder Kaur, Taylors Hill (AU); Delphine Elise Michelle Vincent, Epping (AU); Holly Hone, Parkville (AU); Timothy Ivor Sawbridge, Coburg (AU); Ross Mann, Coburg (AU); German Carlos Spangenberg, Bundoora (AU)

(73) Assignee: Agriculture Victoria Services PTY LTD, Bundoora, Victoria (AU)

(21) Appl. No.: 17/627,776

(22) PCT Filed: Jul. 17, 2020
(86) PCT No.: PCT/AU2020/050733

§ 371 Date: Jan. 17, 2022

(30) Foreign Application Priority Data
Jul. 19, 2019 (AU) 2019902563
Nov. 8, 2019 (AU) 2019904226

(51) Int. Cl. C12Q 1/04

(52) U.S. Cl. CPC C12Q 1/04; C12R 2001/01

(57) ABSTRACT

The present invention provides a method for profiling plant endophyte microbiomes, wherein the method provides for identification of endophyte strains that are phylogenetically related to a desired endophyte strain. More particularly, the present invention relates to method for identifying, characterising and/or comparing endophyte strains and to novel endophyte strains selected and/or isolated by the method. The present invention also relates to methods for transferring endophyte strains between plants.

### FIELD OF THE INVENTION

The present invention relates to method for identifying, characterising and/or comparing endophyte strains and to novel endophyte strains selected and/or isolated by the method. The present invention also relates to methods for transferring endophyte strains between plants.

### BACKGROUND OF THE INVENTION

Microbes represent an invaluable source of novel genes and compounds that have the potential to be utilised in a range of industrial sectors. Scientific literature gives numerous accounts of microbes being the primary source of antibiotics, immune-suppressants, anticancer agents and cholesterol-lowering drugs, in addition to their use in environmental decontamination and in the production of food and cosmetics.

A relatively unexplored group of microbes known as endophytes, which reside e.g. in the tissues of living plants, offer a particularly diverse source of novel compounds and genes that may provide important benefits to society, and in particular, agriculture.

Endophytes may be fungal or bacterial. Endophytes often form mutualistic relationships with their hosts, with the endophyte conferring increased fitness to the host, often through the production of defence compounds. At the same time, the host plant offers the benefits of a protected environment and nutriment to the endophyte.

Important forage grasses perennial ryegrass (Lolium perenne) are commonly found in association with fungal and bacterial endophytes. However, there remains a general lack of information and knowledge of the endophytes of these grasses as well as of methods for the identification and characterisation of novel endophytes and their deployment in plant improvement programs.

Glycine (soybean) is a genus in the bean family Fabaceae. The best known species is the cultivated soybean (Glycine max). Again, there remains a general lack of information and knowledge of the endophytes of these plants as well as of methods for the identification and characterisation of novel endophytes and their deployment in plant improvement programs.

Knowledge of the endophytes of perennial ryegrass may allow certain beneficial traits to be exploited in enhanced pastures, or lead to other agricultural advances, e.g. to the benefit of sustainable agriculture and the environment.

Identification of phylogenetically related microbes typically involves isolation of microbes from e.g. plant material and subsequent processing by genetic sequencing to allow for comparison of microbes. Microbes can then be clustered based on genetic similarities.

Further methods for identification and characterization of for microbes, in particular endophytes, are generally based on morphological characterisation and molecular taxonomy analyses. Morphological characterisation includes analyses of macroscopic and microscopic structures of microbes grown on culture media. Molecular taxonomy analysis is mainly based on gene sequence analysis of spacer regions in nuclear ribosomal DNA (nrDNA), particularly in phylogenomics. However, traditional methods of phylogenomics based on nrDNA sequences may not reflect the divergence of closely related species.

There exists a need to overcome, or at least alleviate, one or more of the difficulties or deficiencies associated with the prior art.

### SUMMARY OF THE INVENTION

In one aspect, the present invention provides a method for profiling endophyte strains from a microbiome, said method including the steps of:
providing a microbiome;
obtaining protein profile spectra from one or more endophytes of the microbiome;
processing the protein profile spectra;
clustering the endophyte strains based on the processed protein profile spectra; and
selecting and/or isolating endophyte strain(s) having desired genetic and/or metabolic characteristics, or being phylogenetically related to a desired endophyte strain.

As used herein, the term ‘profiling’ endophyte strain(s) means identifying, characterising and/or comparing endophyte strain(s). For example, this may include selecting and/or isolating endophyte strain(s) having desired genetic and/or metabolic characteristics, or endophyte strain(s) that are phylogenetically related to a desired endophyte strain.

As used herein the term ‘isolated’ means that an endophyte is removed from its original environment (e.g. the natural environment if it is naturally occurring). For example, a naturally occurring endophyte present in a living plant is not isolated, but the same endophyte separated from some or all of the coexisting materials in the natural system, is isolated.

As used herein the term ‘endophyte’ is meant a bacterial or fungal strain that is closely associated with a plant. By ‘associated with’ in this context is meant that the bacteria or fungus lives on, in or in close proximity to a plant. For example, it may be endophytic, for example living within the internal tissues of a plant, or epiphytic, for example growing externally on a plant.

In a preferred embodiment the microbiome may be isolated from a plant material. The plant material may be of any suitable type. For example, the plant material may be from a grass, tree, flower, herb, shrub or bush, vine or legume, or a product thereof. The method according to the present invention is particularly applicable to grasses and legumes.

In a preferred embodiment the plant material may be from a perennial ryegrass (Lolium perenne), tall fescue (Festuca arundinaceae), corn (Zea mays), Glycine species, wheat (Triticum aestivum) and barley (Hordeum vulgare), or any combination thereof.

In a further preferred embodiment the Glycine species includes Glycine tomentella, Glycine tabacina, Glycine latifolia, Glycine hirticaulis, Glycine microphylla, Glycine clandestine.

In another preferred embodiment the Glycine species includes Glycine max.

In a preferred embodiment the plant material includes seeds, leaves, stems, petioles, roots, buds, flowers or any combination thereof.

In a preferred embodiment, the step of providing the microbiome includes the steps of:
providing plant material;
washing the plant material in an aqueous solution;
submerging the plant material in an aqueous solution;
macerating the plant material; and
applying the macerated plant material to a growth medium for growth of the microbiota to provide the isolated microbiome.

In a preferred embodiment, the microbiota grown on the growth medium may be subjected to a re-streaking so as to obtain an isolated endophyte colony.

In a preferred embodiment, the method may include obtaining protein profile spectra from one or more isolated endophyte colonies.

In a preferred embodiment, when the plant material includes a seed, the step of providing the microbiome may include the preliminary steps of:
harvesting the plant material;
sterilising the plant material;
germinating the plant material;
growing the germinated plant material.

In a further preferred embodiment, the aqueous solution may be a buffer solution. In a further preferred embodiment the buffer solution may be a phosphate buffered saline (PBS) solution.

In a preferred embodiment, the protein profile spectra may be obtained by mass spectrometry. In a further preferred embodiment, the mass spectrometry technique used to obtain the protein profile spectra may be matrix assisted laser desorption/ionisation (MALDI) mass spectrometry.

In a preferred embodiment, the protein profile spectra are processed by a data deconvolution workflow. In a further preferred embodiment the data deconvolution workflow includes performing the steps of:
a m/z scan to create a m/z grid;
a spectrum baseline subtraction; and
a m/z alignment;
   wherein the data deconvolution workflow provides the processed protein spectra.

In a preferred embodiment, processed protein profile spectra may be obtained for one or more isolated endophyte colonies.

As used herein ‘m/z’ means a measurement of the mass to charge ratio resulting from a mass analysis experiment wherein one or more electrons are taken from molecules to create charged ions. The number of electrons removed is the charge number (for positive ions), wherein m/z represents mass (m) divided by charge number (z).

In a further preferred embodiment the m/z grid may be produced according to an adaptive grid method. In a more preferred embodiment the adaptive grid method scan count is between approximately 1 and 10.

In a further preferred embodiment the spectrum baseline subtraction may be performed according to a quantile normalization method. In a more preferred embodiment the quantile normalization method may be limited to between approximately 10% and 20%. In a more preferred embodiment the spectrum baseline subtraction may be performed wherein the m/z window is between approximately 10 and 100 Da.

In a preferred embodiment the m/z alignment may be performed with reference to a reference spectrum. In a further preferred embodiment the reference spectrum may be from Escherichia coli ATCC 25922. In a further preferred embodiment the m/z alignment may be performed with a spectrum index between approximately 1 and 5. In a further preferred embodiment the m/z alignment may be performed with a m/z window between approximately 5 and 1000 Da. In a further preferred embodiment the m/z alignment may be performed wherein the maximum m/z shift is between approximately 1 and 200 Da.

In a preferred embodiment, the method includes combining the protein profile spectra, for example protein profile spectra from one or more isolated endophyte colonies, and performing one or more of the steps of:
a m/z alignment;
a spectrum smoothing;
a m/z range restriction;
a spectrum peak detection; and
a valid peak filtration to remove peaks which do not meet a defined threshold.

In a preferred embodiment, the processed protein profiles may be converted into a matrix for analysis.

In a further preferred embodiment the m/z ratio alignment may be performed with reference to a reference spectrum. In a further preferred embodiment the m/z ratio alignment may be performed with reference to a reference spectrum from Escherichia coli ATCC 25922.

In a further preferred embodiment the m/z ratio alignment may be performed with a spectrum index between approximately 1 and 5. In a further embodiment spectrum smoothing may be performed according to a moving average algorithm. In a further preferred embodiment spectrum smoothing may be performed with a m/z window of approximately 4 to 30 points.

In a further preferred embodiment the m/z range may be between approximately 2000 and 20000 Da.

In a further preferred embodiment the spectrum peak detection may be performed by a resolution-based method. In a further preferred embodiment the boundary determination may be performed at maximum curvature peak filtering. In a further preferred embodiment the spectrum peak detection may be present in at least two experiments. In a further preferred embodiment the valid feature filter may have a threshold between approximately 0-40% intensity. In a further preferred embodiment the feature filter may be present in at least 2 experiments.

In a further embodiment the processed protein profile spectra may be used to perform hierarchal clustering. In a further embodiment the hierarchal clustering may be used to compare endophyte strains and, in particular, identify related endophyte strains.

In a preferred embodiment the hierarchal clustering provides a phenogram wherein endophyte strains are clustered based on similar protein profiles. In a further embodiment the hierarchical clustering provides a clade of endophytes having properties selected from:
i. related bioactivity;
ii. related geographic ranges; or
iii. belonging to plant lines having the same phenotype.

In another preferred embodiment the hierarchical clustering provides a clade of endophytes having properties selected from:
i. related bioactivity;
ii. related geographic ranges;
iii. belonging to plant lines having the same phenotype; or
iv. similar protein profiles.

In a further embodiment the related bioactivity may be selected from bioprotection and biofertilizer activity. In a preferred embodiment the bioprotection and biofertilizer properties may be the same or similar to those of Xanthomonas sp. bacterial strain GW.

By “bioactivity” or “bioactive properties” is meant the capacity of a compound to elicit pharmacological or toxicological effects in plants, humans or animals. In particular, plants may contain secondary compounds and metabolites with bioactive properties that are produced by endophytes.

For example, the bioactivity may be selected from bioprotection and biofertilizer activity. In a preferred embodiment the bioprotection and biofertilizer properties may be the same or similar to those of Xanthomonas sp. bacterial strain GW.

As used herein the term ‘bioprotection and/or biofertilizer’ means that the endophyte possesses genetic and/or metabolic characteristics that result in a beneficial phenotype in a plant harbouring, or otherwise associated with, the endophyte. Such beneficial properties include improved resistance to pests and/or diseases, improved tolerance to water and/or nutrient stress, enhanced biotic stress tolerance, enhanced drought tolerance, enhanced water use efficiency, reduced toxicity and enhanced vigour in the plant with which the endophyte is associated, relative to an organism not harbouring the endophyte or harbouring a control endophyte such as standard toxic (ST) endophyte.

The pests and/or diseases may include, but not limited to, fungal and bacterial pathogens. In a particularly preferred embodiment, the endophyte may result in the production of the bioprotectant compound in the organism with which it is associated.

As used herein, the term ‘bioprotectant compound’ means a compound that provides bioprotection to the plant or aids the defence of the plant with which it is associated against pests and/or diseases, such as fungal and/or bacterial pathogens. A bioprotectant compound may also be known as a ‘biocidal compound’. In a particularly preferred embodiment, the endophyte produces a bioprotectant compound and provides bioprotection to the organism against fungal and/or bacterial pathogens. The terms bioprotectant, bioprotective and bioprotection (or any other variations) may be used interchangeably herein.

As used herein, a ‘bioprotectant property’ provides bioprotection to the plant or aids the defence of the plant against pests and/or diseases, such as fungal and/or bacterial pathogens. A bioprotectant compound may also be known as a ‘biocidal compound’. In a particularly preferred embodiment, the endophyte produces a bioprotectant compound and provides bioprotection to the plant with which it is associated against fungal and/or bacterial pathogens. The terms bioprotectant, bioprotective and bioprotection (or any other variations) may be used interchangeably herein.

As used herein, a ‘biofertilizer’ improves the availability of nutrients to the plant with which the endophyte is associated, including but not limited to improved tolerance to nutrient stress.

The nutrient stress may be lack of or low amounts of a nutrient such as phosphate and/or nitrogen. The endophyte is capable of growing in conditions such as low nitrogen and/or low phosphate and enable these nutrients to be available to the plant with which the endophyte is associated.

In a further embodiment there is provided a method for clustering endophytes of the plant phenotype is for drought tolerance or drought resistance.

In a further embodiment the hierarchical clustering provides identification of a clade of endophytes of a group selected from:
i. multiple host species, wherein said host species is from multiple geographic locations;
ii. multiple host species, wherein said host species is from a single geographic location;
iii. one host species, wherein said host species is from multiple geographic locations; or
iv. one host species, wherein said host species is from a single geographic location.

In another aspect, the present invention provides a substantially purified or isolated endophyte strain, preferably selected and/or isolated using a method according to the present invention, as described herein.

In another aspect of the present invention there is provided a method for profiling a plant microbiome said method including the steps of:
providing plant material from a first plant species and plant material from a related second plant species;
characterising the microbiome of the first and second plant species by analysing the plant material; and
assessing the microbiome of the first and second plant species to identify endophyte strains found in both the first and second plant species or endophyte strains found in the second plant species but not in the first plant species.

In a preferred embodiment, the method of profiling a plant microbiome as described herein and the method of profiling endophyte strains from a microbiome as described herein may be performed sequentially.

In a preferred embodiment the first plant species may be selected from perennial ryegrass (Lolium perenne), tall fescue (Festuca arundinaceae), corn (Zea mays), Glycine species, wheat (Triticum aestivum) and barley (Hordeum vulgare).

In a further preferred embodiment the first plant species may be selected from Glycine tomentella, Glycine tabacina, Glycine latifolia, Glycine hirticaulis, Glycine microphylla, Glycine clandestine and Glycine Max.

In a further embodiment the second plant species may be a crop wild relative (CWR) of the first plant species.

As used herein ‘crop wild relative (CWR)’ is meant plants that have not been domesticated and are genetically related to crop plants. These CWR may be used as a source of endophytes or alleles for crop plants that have been lost through their domestication. Accordingly, in a preferred embodiment, the present invention may include screening microbiomes of CWR, such as Glycine CWR, to identify endophytes that could be substituted for similar endophytes of the crop plant, or introduced into the crop plant, for example the crop plant Glycine max (soybean).

In a further embodiment providing the plant material from the first and second plant species includes the steps of:
sterilising the plant material; and
germinating the plant material,
   wherein the germinated plant material provides seedlings for characterisation.

In a further preferred embodiment, the step of providing the plant material may include the steps of:
washing the plant material in an aqueous solution;
submerging the plant material in an aqueous solution;
macerating the plant material; and
applying the macerated plant material to a growth medium for growth of the microbiota to provide an isolated microbiome.

In a further preferred embodiment, when the plant material includes a seed, the step of providing the plant material may include the preliminary steps of:
harvesting the plant material;
sterilising the plant material;
germinating the plant material; and/or
growing the germinated plant material.

In a preferred embodiment the plant material may be from a grass, tree, flower, herb, shrub or bush, vine or legume, or a product thereof. The method according to the present invention is particularly applicable to grasses and legumes. In a further preferred embodiment the plant material may be selected from seeds, leaves, stems, petioles, roots, buds, flowers or any combination thereof.

In a further preferred embodiment, the aqueous solution may be a buffer solution. In a further preferred embodiment the buffer solution may be a phosphate buffered saline (PBS)

### Solution

In a further embodiment characterising the microbiome of the first and second plant species includes the steps of:
extracting nucleic acid from the plant material; and
analysing the extracted nucleic acid to profile the plant material microbiome.

The steps of extracting nucleic acid and analysing the extracted nucleic acid as used herein may be performed by any suitable technique. In a particularly preferred embodiment analysis of the extracted nucleic acid may be performed by Unweighted UNIFRAC Distance Principal Components Analysis utilising operational taxomic units (OTU) phylogeny.

In a preferred embodiment, the microbiota grown on the growth medium may be subjected to a re-streaking so as to obtain an isolated endophyte colony.

In a preferred embodiment, the method may include obtaining protein profile spectra from one or more isolated endophyte colonies.

In a further preferred embodiment, the step of assessing the microbiome of the first and second plant species may include statistical analysis to determine microbiome operational taxomic units (OTU) present within each plant species.

As used herein ‘operational taxomic units (OTU)’ may include clusters of related organisms having similar physiological, bioactivity, genetic or peptide properties.

In a further preferred embodiment the microbiome OTU within the first plant species may be compared with that of the second plant species to determine microbiota which are either:
shared between the plant species, or
unique to the second plant species, relative to the first plant species.

In a preferred embodiment the identified microbiome OTU may be selected from the group including Stenotrophomonas sp., Pseudomonas sp., Acinetobacter sp., Holomonas sp., Enterobactereaceae sp., Pantoea sp., Burkholderiaceae sp., Ralstonia sp., Massilia sp., Herbaspirillum sp., Delftia sp., Curvibacter sp., Aquabacterium sp., Sphingomonas sp., Novosphingobium sp., Bradyrhizobium sp., Ochrobactrum sp., Methylobacterium sp., Lactobacillus sp., Staphylococcus sp., Bacillus sp. and Curtobacterium sp.

In a further aspect of the present invention, there is provided a method for enhancing the bioactivity of a plant species, said method including:
identifying endophyte strains found in the first and second plant species; and
transferring one or more endophyte strains from the second plant species to the first plant species to enhance bioactivity of the first plant species,
   wherein the transferred endophyte enhances bioactivity of the first plant species.

In a preferred embodiment, the step of identifying endophyte strains found in the first and second plant species may be performed by a method of profiling plant microbiome, as hereinbefore described.

In a further aspect of the present invention, there is provided a method for enhancing the bioactivity of a plant species, said method including:
identifying endophyte strains found in the second plant species but not in the first plant species, and
transferring one of said more endophyte strains from the second plant species to the first plant species,
   wherein the transferred endophyte enhances bioactivity of the first plant species.

In a preferred embodiment, the step of identifying endophyte strains found in the second plant species but not in the first plant species may be performed by a method of profiling plant microbiome, as hereinbefore described.

By “enhancing the bioactivity of a first plant species” is meant that the first plant species has a beneficial phenotype relative to the same plant species not harbouring the transferred endophyte or harbouring a control endophyte such as standard toxic (ST) endophyte. Such beneficial properties include improved resistance to pests and/or diseases, improved tolerance to water and/or nutrient stress, enhanced biotic stress tolerance, enhanced drought tolerance, enhanced water use efficiency, reduced toxicity and enhanced vigour in the plant with which the endophyte is associated, relative to a plant not harbouring the endophyte or harbouring a control endophyte such as standard toxic (ST) endophyte.

In a preferred embodiment the microbiome may be isolated from plant material of the first and second plant species. The plant material may be of any suitable type. In a preferred embodiment the plant material includes seeds, leaves, stems, petioles, roots, buds, flowers or any combination thereof.

By a “related second plant species” is meant a plant species sharing substantial genetic identity with the first plant species, for example greater than approximately 80% genetic identity, preferably greater than 90% genetic identity, more preferably greater than approximately 95% genetic identity, even more preferably greater than approximately 98% genetic identity.

In a preferred embodiment, the protein profile spectra may be obtained by mass spectrometry. In a further preferred embodiment, the mass spectrometry technique used to obtain the protein profile spectra may be matrix assisted laser desorption/ionisation (MALDI) mass spectrometry, as hereinbefore described.

The step of transferring one or more of said endophyte strains from the second plant species to the first plant species may be performed by any suitable technique. Preferably, the first plant species is infected with the endophyte by a method selected from the group consisting of inoculation, breeding, crossing, hybridization and combinations thereof.

In a further aspect, the present invention provides a plant, plant part or plant product with enhanced bioactivity produced by a method according to the present invention.

In another aspect, the present invention provides a substantially purified or isolated endophyte strain, preferably selected and/or isolated using a method according to the present invention, as described herein.

In a preferred embodiment, according to any aspect of the present invention, the endophyte strain may be a strain of Xanthomonas sp. which provides bioprotection and/or biofertilizer phenotypes to plants into which it is inoculated. In a preferred embodiment, the Xanthomonas sp. strain may be GW as described herein and as deposited with The National Measurement Institute of 1/153 Bertie Street, Port Melbourne, VIC 3207, Australia on 17 May 2019 with accession number V19/009902.

In another preferred embodiment the endophyte strain may be a strain of Xanthomonas sp. which provides enhanced bioactivity to plants into which it is inoculated.

In another preferred embodiment, according to any aspect of the present invention, the endophyte strain may be a strain of Arthrobacter sp. which provides bioprotection and/or biofertilizer phenotypes to plants into which it is inoculated. In a preferred embodiment, the Arthrobacter sp. strain may be a D4-11 strain as described herein and as deposited with The National Measurement Institute of 1/153 Bertie Street, Port Melbourne, VIC 3207, Australia on 9 Jul. 2019 with accession number V19/013680.

In another preferred embodiment the endophyte strain may be a strain of Arthrobacter sp. which provides enhanced bioactivity to plants into which it is inoculated.

In another preferred embodiment, according to any aspect of the present invention, the endophyte strain may be a strain of Papiliotrema sp. which provides bioprotection and/or biofertilizer phenotypes to plants into which it is inoculated. In a preferred embodiment, the Papiliotrema sp. strain may be a strain selected from the group consisting of P2-Gland-NS-Runn Creek IS-107-1, P1-Geland-NS-Mornington-IS-114-1, and P2-Geland-NS-Card Creek IS-34-1 strain as described herein and as deposited with The National Measurement Institute of 1/153 Bertie Street, Port Melbourne, VIC 3207, Australia on 9 Jul. 2019 with accession numbers V19/013679, V19/013678 and V19/013677, respectively.

In another preferred embodiment the endophyte strain may be a strain of Papiliotrema sp which provides enhanced bioactivity to plants into which it is inoculated.

In another preferred embodiment, according to any aspect of the present invention, the endophyte strain may be a strain of Sphingomonas paucimobilis which provides bioprotection and/or biofertilizer phenotypes to plants into which it is inoculated. In a preferred embodiment, the Sphingomonas paucimobilis strain may be Gtom-P2-19 as described herein and as deposited with The National Measurement Institute of 1/153 Bertie Street, Port Melbourne, VIC 3207, Australia on 9 Jul. 2019 with accession number V19/013676.

In another preferred embodiment the endophyte strain may be a strain of Sphingomonas paucimobilis which provides enhanced bioactivity to plants into which it is inoculated. In another preferred embodiment, according to any aspect of the present invention, the endophyte strain may be a strain of Argobacterium lanymoorei which provides bioprotection and/or biofertilizer phenotypes to plants into which it is inoculated. In a preferred embodiment, the Argobacterium lanymoorei strain may be a strain selected from Gcla-P1-10, and Gtab-P2-18 as described herein and as deposited with The National Measurement Institute of 1/153 Bertie Street, Port Melbourne, VIC 3207, Australia on 9 Jul. 2019 with accession numbers V19/013675 and V19/013671, respectively.

In another preferred embodiment the endophyte strain may be a strain of Argobacterium lanymoorei which provides enhanced bioactivity to plants into which it is inoculated.

In another preferred embodiment, according to any aspect of the present invention, the endophyte strain may be a strain of Pseudomonas oryzihabitans which provides bioprotection and/or biofertilizer phenotypes to plants into which it is inoculated. In a preferred embodiment, the Pseudomonas oryzihabitans strain may be a strain selected from Ghir-A-22-2, Gtab-P2-12 and Gtom-P2-14 as described herein and as deposited with The National Measurement Institute of 1/153 Bertie Street, Port Melbourne, VIC 3207, Australia on 9 Jul. 2019 with accession numbers V19/013672, V19/013674 and V19/013673, respectively.

In another preferred embodiment the endophyte strain may be a strain of Pseudomonas oryzihabitans which provides enhanced bioactivity to plants into which it is inoculated.

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE DRAWINGS/FIGURES

In the Figures:

FIG. 1 shows the data deconvolution workflow and associated parameters (Refiner, Genedata) used to process raw MALDI spectra from multiple novel bacterial strains across multiple Batches, in order to align and smooth spectra, reduce background noise, and identify all valid protein peaks and their relative intensities.

FIG. 2 shows a region of the Hierarchical Clustering tree generated from protein spectra from novel bioactive bacterial strain GW and other Xanthomonas strains. The novel Xanthomonas sp. bacterial strain GW_0_F7_1 (V19/009902, star) is a representative strain of this clade (bioactive strain)

FIG. 3 shows the protein spectra of the three Xanthomonas sp. novel bacterial strains GW, SS and SI. A. Full chromatogram (2000-14000 Daltons). Proteins unique to either the novel bacteria strain GW (4646.0561) or SS (4494.1438) are evident within the boxed area.

FIG. 4 shows a region of the Hierarchical Clustering tree generated from protein spectra from novel Pseudomonas oryzihabitans bacterial strains isolated from three Glycine species, Glycine tomentella (Gtom designation), Glycine hirticaulis (Ghir designation) and Glycine tabacina (Gtab designation) from two locations, Kakadu National Park (NT), La Trobe University Wildlife Sanctuary (Victoria). The novel Pseudomonas oryzihabitans bacterial strains Ghir-A-22-2 (V19/013672, star) and Gtom-P2-14 (V19/013673, star) and Gtab-P2-12 (V19/013674, star) are representative strains of this clade (broad host range, broad geographic range).

FIG. 5 shows a region of the Hierarchical Clustering tree generated from protein spectra from novel Agrobacterium lartymoorei bacterial strains isolated from two Glycine species, Glycine clandestina (Gcla designation) and Glycine tabacina (Gtab designation) from one location, the La Trobe University Wildlife Sanctuary (Victoria). The novel Agrobacterium lartymoorei bacterial strains Gtab-P2-18 (V19/013671, star) and Gcla-P1-10 (V19/013675, star) are representative strains of this clade (broad host range, narrow geographic range).

FIG. 6 shows a region of the Hierarchical Clustering tree generated from protein spectra from fungal strains (Papiliotrema sp.) isolated from only Glycine clandestina from three locations, Mornington Peninsula National Park (Mornington designation), Cardinia Creek Parklands (Card_Creek designation) and Kinglake National Park: Running Creek (Runn_creek designation) (Victoria). The novel (Papiliotrema sp.) fungal strains P1-Gcland-NS-Card_Creek-IS-34-1 (V19/013677, star), P1-Gcland-NS-Mornington-IS-114-1 (V19/013678, star) and P2-Gcland-NS-Runn_creek-IS-107-1 (V19/013679, star) are representative strains of this clade (narrow host range, broad geographic range).

FIG. 7 shows a region of the Hierarchical Clustering tree generated from protein spectra from Sphingomonas bacterial strains isolated from only Glycine tomentella from one location, Kakadu National Park (NT). The novel Sphingomonas paucimobilis bacterial strain Gtom-P2-19 (V19/013676, star) is a representative strain of this clade (narrow host range, narrow geographic range).

FIG. 8—Seed microbiome profiles of Glycine clandestina, G. tabacina and G. max, collected from various locations across the greater Melbourne region in Victoria, Australia.

FIG. 9—Sphingomonas sp. clade from a Hierarchical Clustering analysis of MALDI protein spectra from strains isolated from G. clandestina (Gcland designation, light grey star) and G. max (Gmax designation, dark grey star), demonstrating a microbe that could potentially be transferred from one species to another.

FIG. 10 shows a region of the hierarchical tree generated from protein spectra from Arthrobacter bacterial strains isolated from drought tolerant wheat lines. The novel Arthrobacter sp. bacterial strain D4-11 (V19/013680, star) is a representative strain of this clade (associated with a plant phenotype—drought tolerance).

### DETAILED DESCRIPTION OF THE EMBODIMENTS

### Using Matrix Assisted Laser Desorption/Ionisation (MALDI) Mass Spectrometry for Plant Microbiome Profiling

The invention comprises methods for profiling plant bacterial and fungal microbiomes using MALDI. The bacterial and fungal microbiome is isolated from plants, and these microbes are identified and compared using MALDI. The spectra from bacterial and fungal strains are processed using novel filtering settings to produce validated spectra that are used for hierarchical clustering. Strains occupying the same clades of the tree shown to be phylogenetically related.

Applying this method allows the rapid identification of phylogenetically related microbes without the need to further process the microbes beyond their isolation, as whole colonies can be used for the MALDI spectra generation and comparison. The more time consuming and costly DNA sequencing can thus be targeted to microbes of interest.

This method can be used to specifically identify bacterial and fungal strains among a collection of isolated microbes that (i) are closely related to strains with bioactivity (e.g. bioprotection and biofertilizer properties); (ii) are closely related to strains with broad/narrow host ranges from broad/narrow geographic ranges; (iii) are closely related to strains from plant lines with a specific phenotype (e.g. drought tolerance).

### Example 1—Microbe Isolation

Plant material (seeds, leaves, stems, petioles, roots, buds, flowers) was harvested from perennial ryegrass (Lolium perenne), tall fescue (Festuca arundinaceae), corn (Zea mays), Glycine species, wheat (Triticum aestivum) and barley (Hordeum vulgare). Seed was surface sterilised, germinated and allowed to grow for up to 14 days prior to microbe isolation. All other plant material was washed in sterile distilled water or phosphate buffered saline (PBS) up to 5 times prior to microbe isolation. The plant material was submerged in sufficient PBS to completely cover the tissues, and ground using a Qiagen TissueLyser II, for 1 minute at 30 Hertz. A 10 μl aliquot of the macerate was added to 90 μl of PBS. Subsequent 1 in 10 dilutions of the 10⁻¹ suspension were used to create additional 10⁻² to 10⁻⁴ suspensions. Once the suspensions were well mixed 50 μl aliquots of each suspension were plated onto Reasoners 2 Agar (R2A) for growth of bacteria and fungi. Dilutions that provided a good separation of microbial colonies were subsequently used for isolation of individual colonies through re-streaking of single colonies from the dilution plates onto single R2A plates to establish a pure colony.

### Example 2—Spectra Acquisition, Processing and Analysis

### Spectra Acquisition

MALDI spectra were acquired for all novel bacterial and fungal strains to determine the relatedness of each strain. The analysis acquired and compared spectra of protein profiles from each novel bacterial and fungal strain using the Bruker MALDI Biotyper system. Single bacterial and fungal colonies of each strain were generated through streaking from glycerol stocks onto R2A plates and allowing colony growth for 48 hours. Single bacterial and fungal colonies were applied to a Bruker MALDI Biotyper target plate using the Extended Direct Transfer (EDT) method. In the EDT method novel bacterial and fungal strains were inoculated on to two consecutive wells on the target plate (primary spot and secondary spot), treated with 70% formic acid (for up to 30 mins) and covered with HCCA (α-cyano-4-hydroxycinnamic acid) matrix solution [10 mg HCAA in 1 mL of solvent solution: 50% volume μL ACN (acetonitrile), 47.5% volume μL water, and 2.5% volume μL TFA (trifluoroacetic acid)]. The plate was dried at room temperature. Escherichia coli strain ATCC 25922 was included as a quality control. The target plate was analysed in a Bruker MALDI-TOF ultrafleXtreme according to manufacturer's instructions. Protein spectra were calibrated with the Escherichia coli ATCC 25922 quality control strain, and an internal standard. Automated analysis of the raw spectral data was performed by the MALDI BioTyper automation 2.0 software (Bruker Daltonics) using default settings. Protein spectra were compared to MALDI BioTyper library (3,746 spectra—Jun. 9, 2010) for preliminary identification and taxonomical assignment.

### Spectra Processing

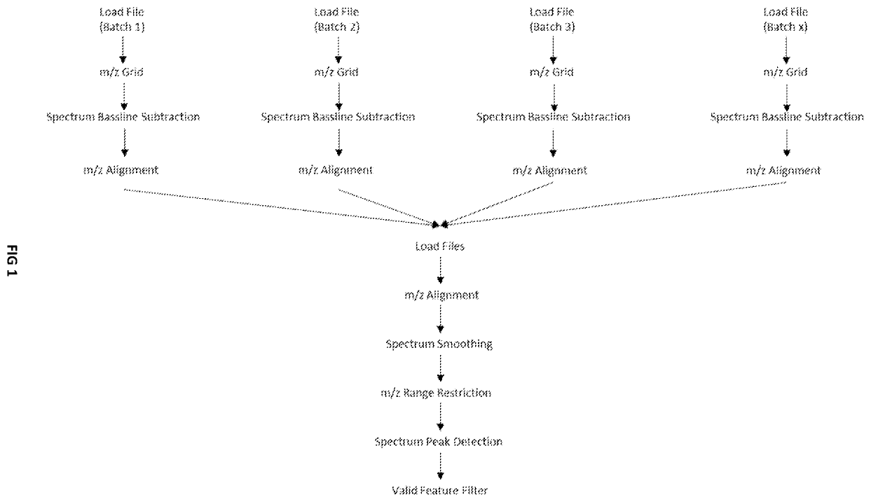

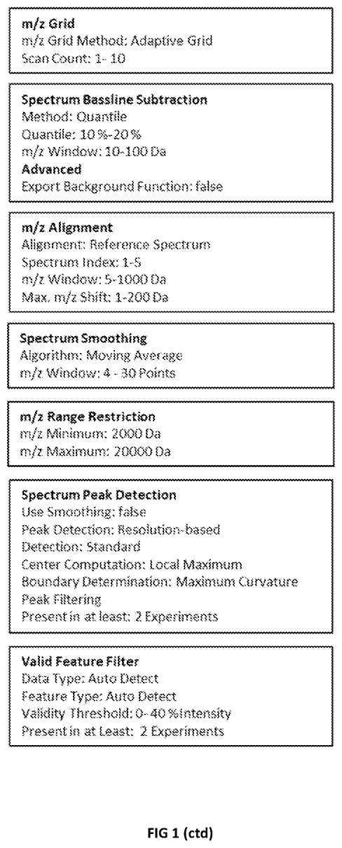

The raw protein spectra from each novel bacterial and fungal strain were processed through a data deconvolution workflow in the software Refiner, GeneData. The raw spectra from each plate (i.e. Batch) were processed separately, first by aligning spectra to create a m/z grid (m/z×sample), followed by spectrum baseline subtraction to reduce background noise across the grid, and finally aligning m/z across key reference spectra from the grid (e.g. E. coli ATCC 25922) (FIG. 1). Batches were then merged and processed further, first by again aligning m/z across key reference spectra (e.g. E. coli ATCC 25922) from the grid, followed by spectrum smoothing to reduce intensity jitter of putative peaks, then restricting m/z from a defined range, then detecting spectrum peaks using a resolution-based method, and finally filtering valid peaks by removing those that did not meet specific thresholds. Parameters for the data deconvolution workflow are defined in FIG. 1. The resultant processed data of valid peaks and intensities was converted into a matrix for statistical analysis.

### Spectra Analysis

The matrix was analysed in the software Analyst, Genedata. A Hierarchical Clustering analysis was conducted to compare protein spectra between novel bacterial and fungal strains. The analysis utilised the Positive Correlation (1-r) distance algorithm, with complete linkage, and only included values present in 50% of samples. A Hierarchical Clustering tree was generated whereby novel bacterial and fungal strains clustered based on similar protein profiles.

### Example 3—Use of MALDI Spectra to Identify Closely Related Bacterial Strains to Bioactive Strains

A method has been developed that uses MALDI to compare protein spectra from bioactive bacterial strains with other bacterial strains isolated from the same host plant, to identify closely related strains that may possess equivalent or superior bioactivity.

A total of 600 bacterial strains were isolated from seed, leaves and roots of perennial ryegrass (Lolium perenne). The novel Xanthomonas sp. bacterial strain GW isolated from seed was demonstrated to have bioprotectant and biofertilizer activity. The protein profiles of all bacterial strains were acquired, processed and analysed to determine their phylogenetic relatedness, including strains that were closely related to the novel bioactive Xanthomonas sp. bacterial strain GW.

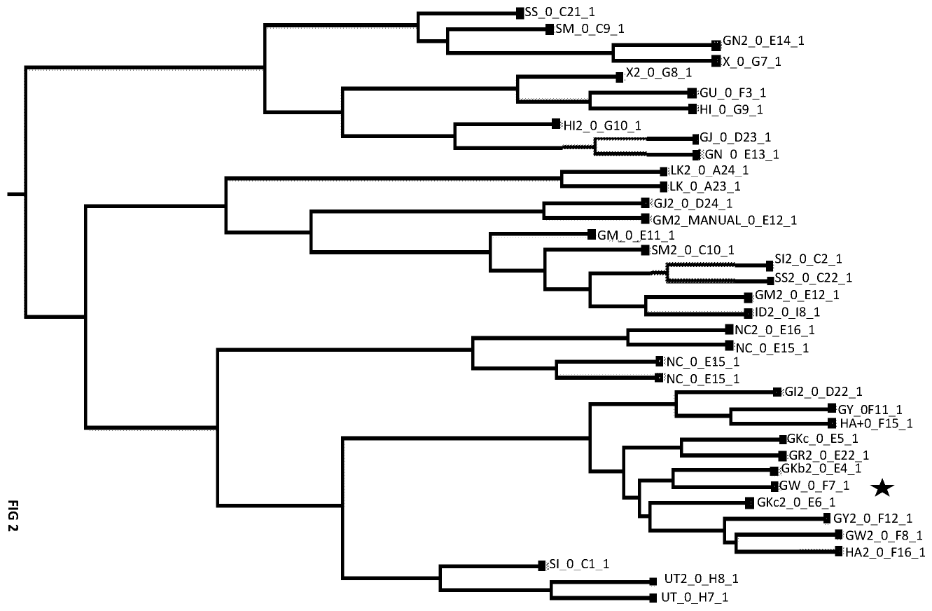

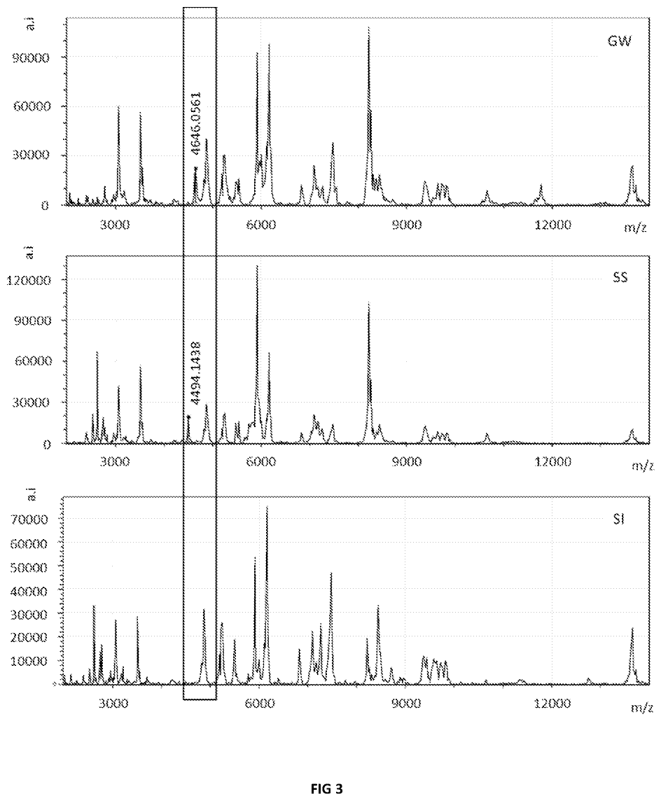

The novel bioactive Xanthomonas sp. bacterial strain GW clustered with 17 additional strains in the phenogram. Some of these strains were isolated from seeds, while others were isolated from leaves and roots from mature plants. These strains included SS, SI, SM, GN, X, GU, HI, GJ, GM, LK, NC, NG, GY, HA, GKc, GKb, UT. The GW containing clade of the Hierarchical Clustering tree was referred to as a Xanthomonas clade (FIG. 2). An assessment of the protein spectra of Xanthomonas sp. strains (GW, SS and SI) indicated that the profiles were highly similar, differing with respect to proteins, 3953.8004 Daltons (unique to strain GW), 4497.4911 Daltons (Unique to strain SS) and 12741.7201 Daltons (Unique to strains SS and SI) (FIG. 3).

The 17 additional strains are candidates for further characterisation to determine if they possess equivalent or superior bioactivity.

### Example 4—Use of MALDI Spectra to Identify Bacterial Strains Common within a Plant Genera (Host Range) and Across Diverse Geographic Locations (Geographic Range)

A method has been developed that uses MALDI to compare protein spectra from bacterial and fungal strains isolated from within a plant genera and from a diverse geographic locations, to identify closely related bacterial and fungal strains that (i) are specific to multiple host species from diverse locations (i.e. broad host range, broad geographic range), (ii) are specific to multiple host species from a single location (i.e. broad host range, narrow geographic range), (iii) are specific to one host species from diverse locations (i.e. narrow host range, broad geographic range) or (iv) are specific to one host species from a single location (i.e. narrow host range, narrow geographic range).

A total of 331 bacterial and fungal strains were isolated from seeds of soybean (Glycine max) and six native Glycine species (Glycine tomentella, Glycine tabacina, Glycine latifolia, Glycine hirticaulis, Glycine microphylla, Glycine clandestina). The protein profiles of all bacterial and fungal strains were acquired, processed and analysed to determine their phylogenetic relatedness.

### Broad Host Range, Broad Geographic Range

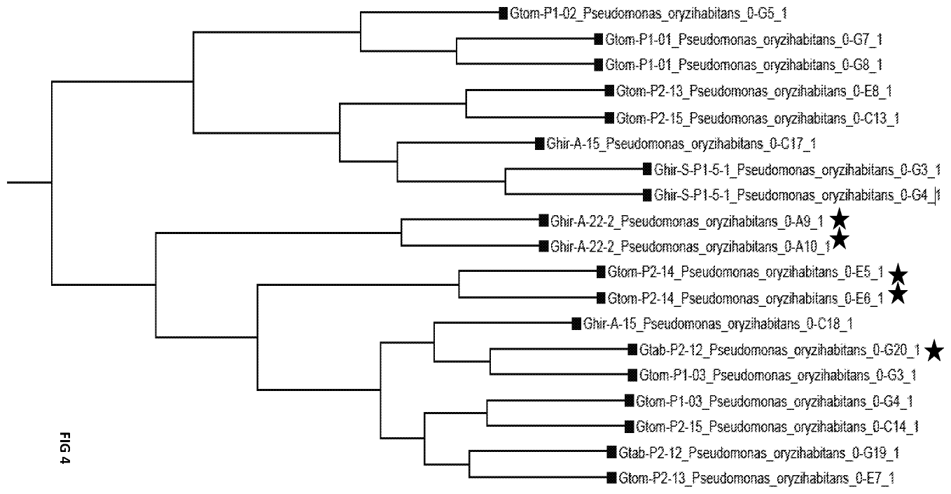

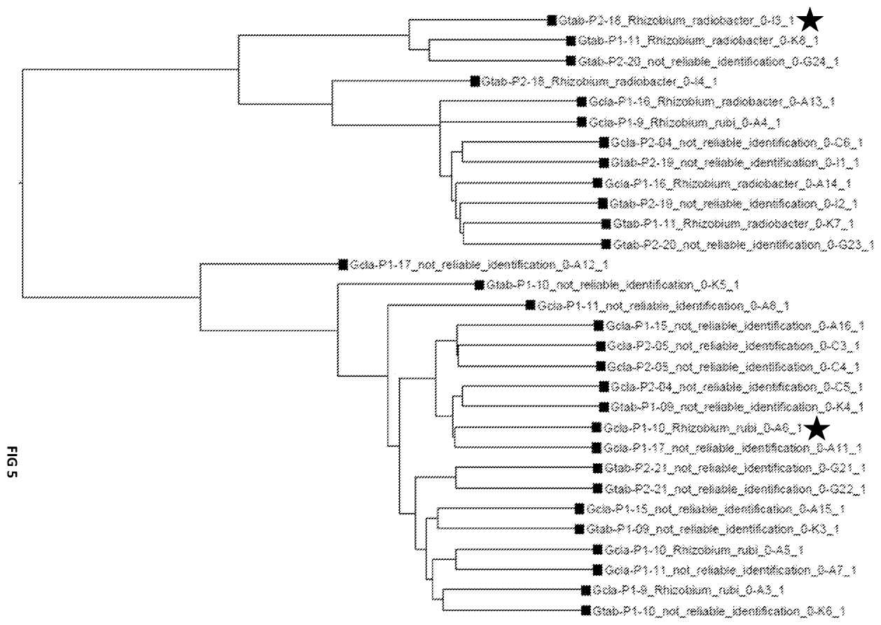

A clade was identified that contained closely related Pseudomonas oryzihabitans bacterial strains, that were isolated from three Glycine species, Glycine tomentella, Glycine hirticaulis and Glycine tabacina (FIG. 4). The bacterial strains were isolated across a range of locations throughout Australia—Kakadu National Park (Northern Territory, NT), La Trobe University Wildlife Sanctuary (Victoria). These bacterial strains all had spectra with identical proteins, differing only in the intensity of those proteins. This clade represents closely related bacterial strains with a broad host range and from a broad geographic range.

### Broad Host Range, Narrow Geographic Range

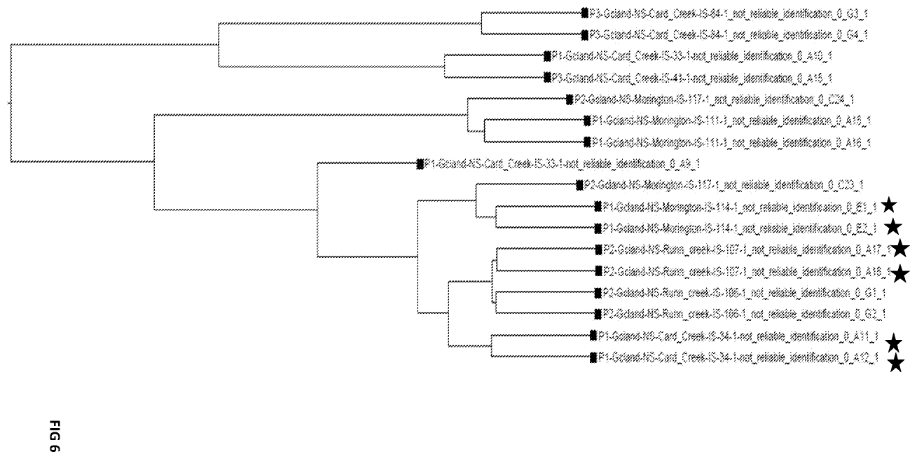

A clade was identified that contained closely related Agrobacterium larrymoorei bacterial strains that were isolated from two Glycine species, Glycine clandestina and Glycine tabacina (FIG. 5). The bacterial strains were isolated from one location in Victoria—La Trobe University Wildlife Sanctuary. These bacterial strains all had spectra with identical proteins, differing only in the intensity of those proteins. This clade represents closely related bacterial strains with a broad host range and from a narrow geographic range.

### Narrow Host Range, Broad Geographic Range

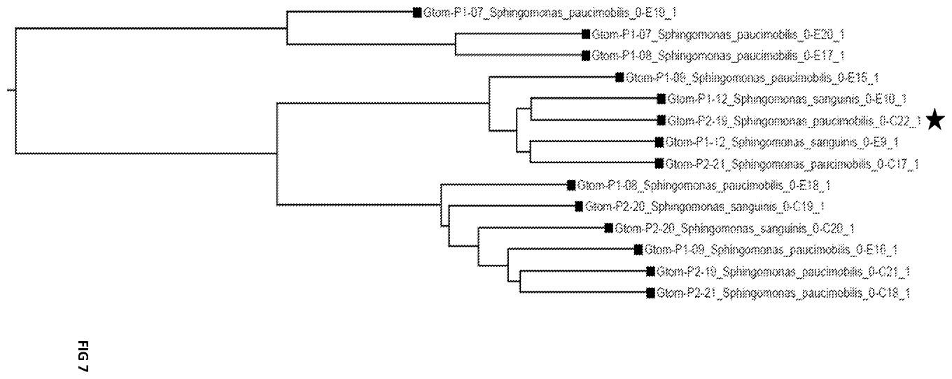

A clade was also identified that contained closely related fungal strains (Papiliotrema sp.), that were isolated from only Glycine clandestina (FIG. 6). The fungal strains were isolated across a range of locations throughout Victoria—Mornington Peninsula National Park, Cardinia Creek Parklands and Kinglake National Park: Running Creek. These fungal strains had spectra with identical proteins, differing only in the intensity of those proteins. This clade represents closely related fungal strains with a narrow host range and from a broad geographic range.

### Narrow Host Range, Narrow Geographic Range

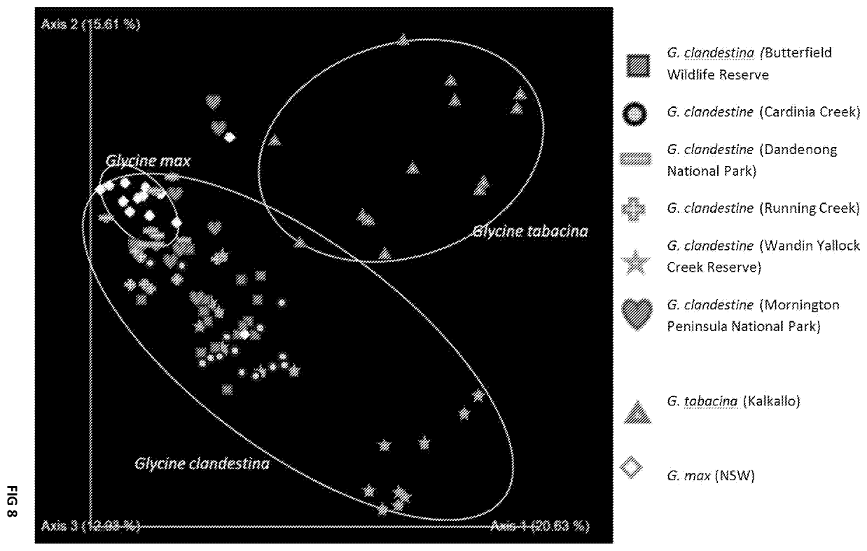

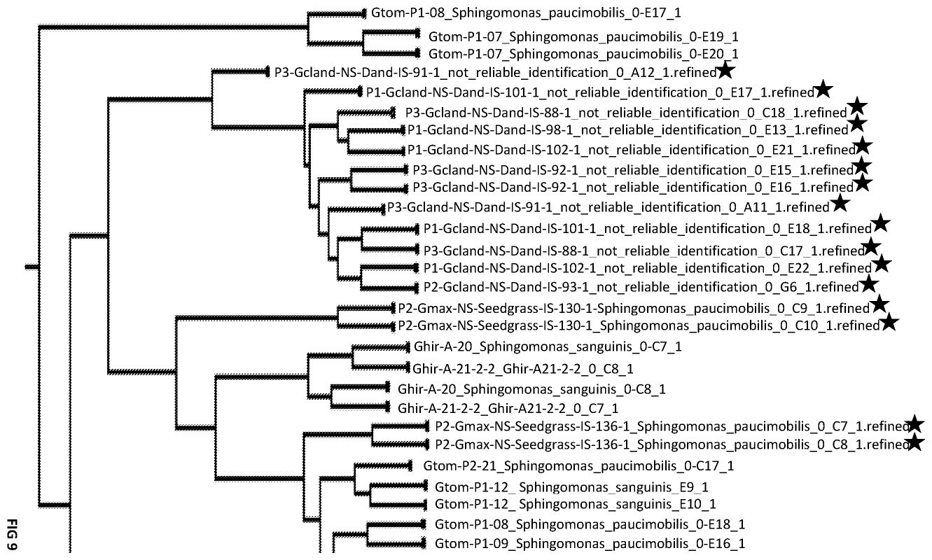

A clade was also identified that contained closely related Sphingomonas bacterial strains that were isolated from only Glycine tomentella (FIG. 7). The bacterial strains were isolated from one location, Kakadu National Park (NT). These bacterial strains all had spectra with identical proteins, differing only in the intensity of those proteins. This clade represents closely related bacterial strains with a narrow host range and from a narrow geographic range.

### Example 5—Use of MALDI to Identify Bacterial Strains Exclusive to a Plant Phenotype

A method has been developed that uses MALDI to compare protein spectra from bacterial strains isolated plant lines exhibiting different phenotypes, to identify closely related bacterial strains that are exclusive to a specific phenotype.

A total of 580 bacterial strains were isolated from seeds of 11 wheat (Triticum aestivum) lines that were phenotyped as either drought tolerant or drought resistant. The protein profiles of all bacterial strains were acquired, processed and analysed to determine their phylogenetic relatedness.

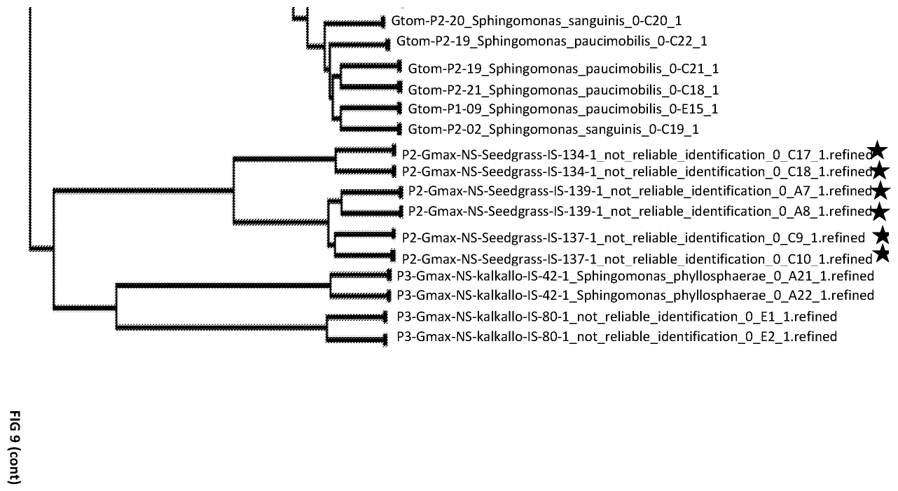

A clade was identified that contained closely related Arthrobacter bacterial strains that were exclusive to the drought tolerant lines (FIG. 8). These bacterial strains all had spectra with identical proteins, differing only in the intensity of those proteins. This clade represents closely related bacterial strains that are exclusive to a plant phenotype.

Finally, it is to be understood that various alterations, modifications and/or additions may be made without departing from the spirit of the present invention as outlined herein.

### Example 6—Profiling the Glycine Seed Microbiome

The seed microbiome of Glycine CWR was compared to commercial G. max to determine similarities across the species. The core microbiome of G. clandestina was identified and compared to G. max to identify operational taxonomic units (OTUs) present in (Scenario I) both Glycine CWRs and G. max, in an effort to identify strains that could be transferred from one species to another, which could potentially offer enhanced bioactivity, or (Scenario II) bacteria that were only found in Glycine CWRs that could be introduced into G. max, which could potentially offer novel bioactivity. The process was validated by isolating strains from Glycine CWRs and G. max, which were taxonomically identified and compared using MALDI-TOF MS to determine if there were strains that clustered according to Scenario I or Scenario II. Candidate microbes were identified that fulfilled Scenario I, and the putative identities of microbes that fulfil criteria were identified within the core microbiome of G. clandestina.

Seed from Glycine max, Glycine clandestina, Glycine hirticaulis, Glycine tomentella, Glycine tabacina, and Glycine microphylla was collected across Victoria and the Northern Territory, and stored at room temperature (22-24° C.). The seed were washed 10 times with excess amount of sterile distilled water in sterile conditions. All the Glycine CWR seeds were then scarified using sterile scalpel blade to initiate the process of water absorption. Seeds of both Glycine max and CWR were germinated in large (12 cm diameter) sterile petri dishes containing three layers of sterile Whatman™ paper (two on bottom and one layer on top of seeds). Under aseptic conditions, 10-20 seeds were placed into each dish followed by the addition of 5-7 ml of sterile distilled water. The petri dishes were sealed with Parafilm™ and incubated first for 2-3 days in dark at room temperature. Once the seed germinated the top layer of filter paper was removed aseptically and petri dishes were then resealed with Parafilm™ allowed further to grow for 10-12 days. Once the seedlings were of sufficient size, a total of 16 seedlings were harvested per Glycine species and per location. DNA extraction of seedlings was performed in 96-well plates using the QIAGEN MagAttract 96 DNA Plant Core Kit according to manufacturers' instructions with minor modifications for use with a Biomek FX liquid handling station. The bacterial microbiome was profiled targeting the V4 region (515F and 806R) of the 16S rRNA gene according to the Illumina 16S Metagenomic Sequencing Library Preparation protocol, with minor modifications to include the use of PNA PCR blockers to reduce amplification of 16S rRNA genes sequences derived from the plant chloroplast genome and mitochondrial genome (Wagner et al., 2016). Paired-end sequencing was performed on HiSeq3000 using a 2×150 bp v3 chemistry cartridge. Sequence data was trimmed and merged using PandaSEQ (removal of low quality reads, 8 bp overlap of read 1 and read 2, removal of primers, final merged read length of 253 bp) (Massela et al., 2012). QIIME2 (release 2019.4) was used for dereplication for taxonomy assignment, removal of organelle OTUs, and statistical analysis (multivariate statistics for qualitative and quantitative OTU analysis; presence/absence searches for core microbiome analysis).

Seed microbiomes were assessed from G. clandestina accessions collected from 6 locations across the greater Melbourne region of Victoria and compared to G. tabacina (one accession—greater Melbourne region) and G. max (one accession—NSVV). The comparison utilised Unweighted UNIFRAC Distance Principal Components Analysis, which is a qualitative assessment that utilises OTU phylogeny (FIG. 8). The microbiomes of G. clandestina and G. max were similar with replicates clustering together, particularly replicates from G. max and G. clandestina (Dandenong National Park), indicating the phylogeny of OTUs within the microbiomes were similar. The seed microbiome of G. tabacina was different from G. max and G. clandestina forming a distinct cluster. An assessment of the core microbiome of G. clandestina identified 22 OTUs present in seed accessions from the six locations, including Stenotrophomonas sp., Pseudomonas sp., Acinetobacter sp., Holomonas sp., Enterobactereaceae sp., Pantoea sp., Burkholderiaceae sp., Ralstonia sp., Massilia sp., Herbaspirillum sp., Delftia sp., Curvibacter sp., Aquabacterium sp., Sphingomonas sp., Novosphingobium sp., Bradyrhizobium sp., Ochrobactrum sp., Methylobacterium sp., Lactobacillus sp., Staphylococcus sp., Bacillus sp. and Curtobacterium sp. (Table 1). Of these, a total of 14 OTUs were also present in G. max, with these OTUs representing microbes that could potentially be transferred between the two species. In addition, there were 8 OTUs that were absent from G. max, with these OTUs representing microbes that could potentially be introduced into G. max. These included Stenotrophomonas sp., Acinetobacter sp., Holomonas sp., Pantoea sp., Curvibacter sp., Aquabacterium sp., Novosphingobium sp. and Bradyrhizobium sp.,

TABLE 1
Core seed microbiome of G. clandestina, and assessment of their presence in G. max. OTUs in bold are
present in both G. clandestina and G. max and represent microbes that could potentially be
transferred between species, while OTUs in underline are only from G. clandestina and represent
microbes that could potentially be introduced into G. max.

| | Glycine clandestina | | | | | | |
|---|---|---|---|---|---|---|---|
| | Butterfield | | Dandenong | Mornington | Running | | Glycine max |
| | Wildlife | Cardinia | Ranges | Peninsula | Creek | Wandin Yallock | New South |
| Bacterial genera | Reserve | Creek | National Park | National Park | Road | Creek Reserve | Wales |
| [symbol] | 0.081% | 0.053% | 20.837% | 1.288% | 0.900% | 0.006% | 0.023% |
| [symbol] | 5.259% | 2.229% | 0.315% | 0.058% | 2.061% | 0.146% | 0.862% |
| [symbol] | 1.507% | 0.198% | 0.014% | 0.024% | 1.242% | 0.021% | 0.020% |
| [symbol] | 0.662% | 0.234% | 0.002% | 1.257% | 1.185% | 0.011% | 0.001% |
| [symbol] | 0.272% | 0.006% | 5.367% | 0.247% | 0.700% | 0.000% | 0.280% |
| [symbol] | 0.253% | 0.177% | 0.024% | 0.549% | 0.155% | 0.001% | 0.019% |
| Bradyrhizobium sp. | 0.051% | 0.006% | 0.023% | 0.584% | 0.088% | 0.001% | 0.000% |
| Novosphingobium sp. | 0.027% | 0.002% | 0.024% | 0.522% | 0.018% | 0.000% | 0.000% |
| [symbol] | 2.309% | 44.282% | 42.417% | 1.514% | 2.075% | 1.791% | 2.132% |
| Aquabacterium sp. | 0.152% | 0.503% | 0.054% | 2.208% | 0.450% | 0.004% | 0.000% |
| Curvibacter sp. | 0.172% | 0.309% | 0.014% | 1.167% | 0.872% | 0.004% | 0.000% |
| [symbol] | 3.926% | 1.781% | 0.627% | 18.702% | 9.746% | 0.018% | 0.224% |
| [symbol] | 0.737% | 0.332% | 0.155% | 1.851% | 0.587% | 0.004% | 0.045% |
| [symbol] | 9.919% | 0.090% | 0.867% | 4.367% | 5.578% | 0.001% | 1.387% |
| [symbol] | 0.845% | 2.128% | 0.247% | 3.509% | 0.812% | 0.005% | 0.043% |
| [symbol] | 0.445% | 0.670% | 0.195% | 5.778% | 5.947% | 0.002% | 0.443% |
| Pantoea sp. | 4.810% | 3.705% | 0.670% | 24.130% | 15.848% | 0.058% | 0.000% |
| [symbol] | 5.621% | 0.780% | 0.537% | 6.228% | 2.276% | 0.010% | 0.093% |
| Halomonas sp. | 0.002% | 0.080% | 0.007% | 0.199% | 0.063% | 0.000% | 0.000% |
| Acinetobacter sp. | 0.191% | 0.480% | 0.034% | 1.854% | 2.764% | 0.010% | 0.000% |
| [symbol] | 52.354% | 35.410% | 1.906% | 18.715% | 13.147% | 97.855% | 43.240% |
| Stenotrophomonas | 0.201% | 0.001% | 0.030% | 0.594% | 0.320% | 0.000% | 0.000% |
| sp. | | | | | | | |

### Example 7—Isolation and Characterisation of the Glycine Microbiome

Seed from the 6 Glycine spp. from Victorian and the Northern Territory were washed and germinated as per Example 6. Seed was harvested by removing aerial tissue and root tissue, and discarding the seed coat. The plant tissues were submerged in Phosphate Buffered Saline (PBS) to cover the plant tissue, and ground using a sterile micropestle or Qiagen TissueLyser II, for 1 minute at 30 Hertz. A 10 μl aliquot of the macerate was added to 90 μl of PBS. Subsequent 1 in 10 dilutions of the 10⁻¹ suspension were used to create additional 10⁻² to 10⁻⁴ suspensions. Once the suspensions were well mixed 20 μl aliquots of each suspension were plated onto Reasoners 2 Agar (R2A) for growth of bacteria. Dilutions that provided a good separation of bacterial colonies were subsequently used for isolation of individual bacterial colonies through re-streaking of single bacterial colonies from the dilution plates onto single R2A plates to establish a pure bacterial colony. Around 400 bacterial strains were obtained from sterile seedlings.

MALDI spectra were acquired for all novel bacterial and fungal strains to determine the relatedness of each strain. The analysis acquired and compared spectra of protein profiles from each novel bacterial and fungal strain using the Bruker MALDI Biotyper system. Single bacterial and fungal colonies of each strain were generated through streaking from glycerol stocks onto R2A plates and allowing colony growth for 48 hours. Single bacterial and fungal colonies were applied to a Bruker MALDI Biotyper target plate using the Extended Direct Transfer (EDT) method. In the EDT method novel bacterial and fungal strains were inoculated on to two consecutive wells on the target plate (primary spot and secondary spot), treated with 70% formic acid (for up to 30 mins) and covered with HCCA (α-cyano-4-hydroxycinnamic acid) matrix solution [10 mg HCAA in 1 mL of solvent solution: 50% volume μL ACN (acetonitrile), 47.5% volume μL water, and 2.5% volume μL TFA (trifluoroacetic acid)]. The plate was dried at room temperature. Escherichia coli strain ATCC 25922 was included as a quality control. The target plate was analysed in a Bruker MALDI-TOF ultrafleXtreme according to manufacturer's instructions. Protein spectra were calibrated with the Escherichia coli ATCC 25922 quality control strain, and an internal standard. Automated analysis of the raw spectral data was performed by the MALDI BioTyper automation 2.0 software (Bruker Daltonics) using default settings. Protein spectra were compared to MALDI BioTyper library (3,746 spectra—Jun. 9, 2010) for preliminary identification and taxonomical assignment. The raw protein spectra from each novel bacterial and fungal strain were processed through a data deconvolution workflow in the software Refiner, GeneData. The raw spectra from each plate (i.e. Batch) were processed separately, first by aligning spectra to create a m/z grid (m/z×sample), followed by spectrum baseline subtraction to reduce background noise across the grid, and finally aligning m/z across key reference spectra from the grid (e.g. E. coli ATCC 25922). Batches were then merged and processed further, first by again aligning m/z across key reference spectra (e.g. E. coli ATCC 25922) from the grid, followed by spectrum smoothing to reduce intensity jitter of putative peaks, then restricting m/z from a defined range, then detecting spectrum peaks using a resolution-based method, and finally filtering valid peaks by removing those that did not meet specific thresholds. The resultant processed data of valid peaks and intensities was converted into a matrix for statistical analysis. The matrix was analysed in the software Analyst, Genedata. A Hierarchical Clustering analysis was conducted to compare protein spectra between novel bacterial and fungal strains. The analysis utilised the Positive Correlation (1-r) distance algorithm, with complete linkage, and only included values present in 50% of samples. A Hierarchical Clustering tree was generated whereby novel bacterial and fungal strains clustered based on similar protein profiles.

The Hierarchical Clustering tree was assessed for clades that contained strains (i) common to both G. clandestine and G. max that could be transferred to both species, and (ii) unique to G. clandestine that could be introduced to G. max. A clade was identified containing Sphingomonas sp. strains isolated from both G. clandestine and G. max, which is a bacterial species identified in the core microbiome of G. clandestine and G. max (FIG. 9).

### Example 8—Use of MALDI to Identify Bacterial Strains Exclusive to a Plant Phenotype

A method has been developed that uses MALDI to compare protein spectra from bacterial strains isolated plant lines exhibiting different phenotypes, to identify closely related bacterial strains that are exclusive to a specific phenotype.

A total of 580 bacterial strains were isolated from seeds of 11 wheat (Triticum aestivum) lines that were phenotyped as either drought tolerant or drought resistant. The protein profiles of all bacterial strains were acquired, processed and analysed to determine their phylogenetic relatedness.

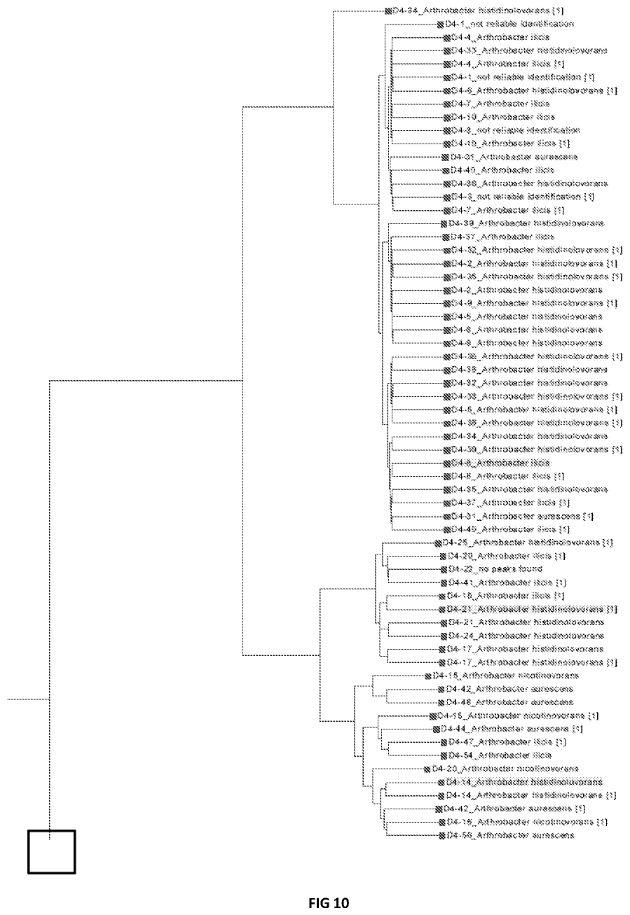

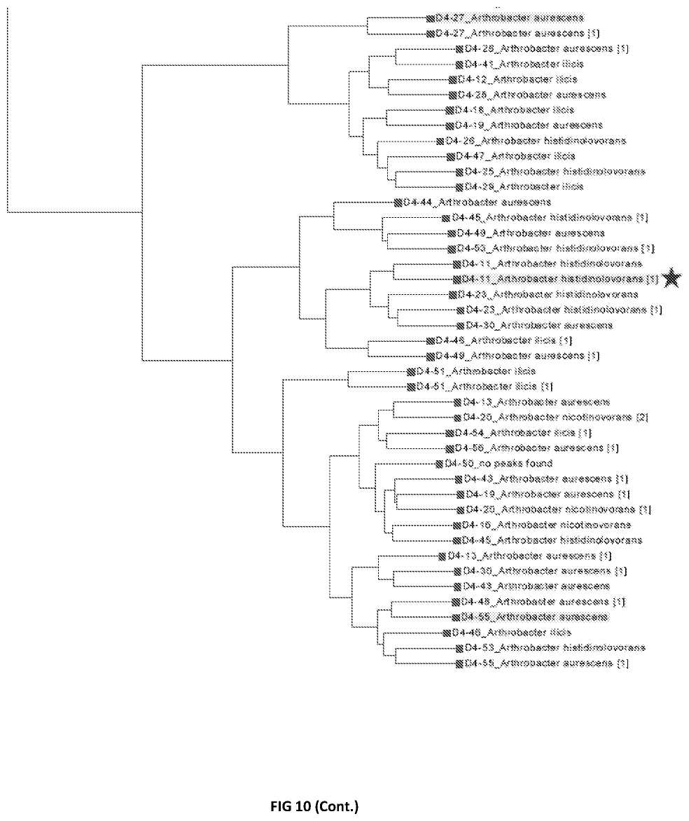

A clade was identified that contained closely related Arthrobacter bacterial strains that were exclusive to the drought tolerant lines (FIG. 10). These bacterial strains all had spectra with identical proteins, differing only in the intensity of those proteins. This clade represents closely related bacterial strains that are exclusive to a plant phenotype.

Finally, it is to be understood that various alterations, modifications and/or additions may be made without departing from the spirit of the present invention as outlined herein.

1-59. (canceled)

60. A method for profiling endophyte strains from a microbiome, said method including the steps of:
providing a microbiome, preferably wherein the microbiome is isolated from a plant material selected from seeds, stems, leaves, petioles, roots, buds, flowers or any combination thereof, preferably isolated from a plant selected from perennial ryegrass (Lolium perenne), tall fescue (Festuca arundinaceae), corn (Zea mays), Glycine species including Glycine max, Glycine tomentella, Glycine tabacina, Glycine latifolia, Glycine hirticaulis, Glycine microphylla, and Glycine clandestine, wheat (Triticum aestivum) and barley (Hordeum vulgare) and preferably wherein the microbiome includes bacteria and/or fungi;
obtaining protein profile spectra from one or more endophytes of the microbiome;
processing the protein profile spectra;
clustering the endophyte strains based on the processed protein profile spectra; and
selecting and/or isolating endophyte strain(s) having desired genetic and/or metabolic characteristics, or being phylogenetically related to a desired endophyte strain.

61. The method according to claim 60, wherein the step of providing the microbiome includes the steps of:
providing plant material;
washing the plant material in an aqueous solution;
submerging the plant material in a aqueous solution;
macerating the plant material; and
applying the macerated plant material to a growth medium for growth of the microbiota to provide the isolated microbiome, preferably wherein the microbiota grown on the growth medium are subjected to a re-streaking so as to obtain an isolated endophyte colony.

62. The method according to claim 61, wherein the plant material includes a seed, and wherein the step of providing the microbiome includes the preliminary step of:
harvesting the plant material;
sterilising the plant material;
germinating the plant material; and
growing the germinated plant material.

63. The method according to claim 60, wherein the protein profile spectra are obtained by mass spectrometry, preferably matrix assisted laser desorption/ionisation (MALDI) mass spectrometry.

64. The method according to claim 60, wherein the protein profile spectra are processed by a data deconvolution workflow, preferably wherein the data deconvolution workflow includes performing the steps of:
a m/z scan to create a m/z grid;
a spectrum baseline subtraction; and
a m/z alignment;
wherein the data deconvolution workflow provides the processed protein spectra.

65. The method according to claim 64, wherein the m/z grid is produced according to an adaptive grid method, the spectrum baseline subtraction is performed according to a quantile normalization method and wherein the m/z alignment is performed with reference to a reference spectrum, preferably wherein the reference spectrum is of Escherichia coli ATCC 25922.

66. The method according to claim 60, wherein protein profile spectra are obtained from one or more isolated endophyte colonies and wherein processing the protein profiles includes combining the protein profile spectra of each isolated endophyte colony.

67. The method according to claim 66 further including subsequently performing the steps of:
m/z alignment;
spectrum smoothing;
m/z range restriction;
spectrum peak detection; and
valid peak filtration to removing peaks which do not meet a defined threshold.

68. The method according to claim 67, wherein the processed protein profiles are converted into a matrix for analysis, wherein the m/z ratio alignment is performed with reference to a reference spectrum, preferably wherein the reference spectrum is of Escherichia coli ATCC 25922, wherein the spectrum smoothing is performed according to a moving average algorithm, wherein the m/z range is restricted to between 2000 Da and 20000 Da, wherein the spectrum peak detection is performed by a resolution-based method, wherein the valid feature filter has a threshold between approximately 0-40% intensity, and wherein processed protein profile spectra are used to perform hierarchal clustering.

69. The method according to claim 68, wherein said hierarchical clustering provides a clade of endophytes having properties selected from:
i. related bioactivity;
ii. related geographic ranges;
iii. belonging to plant lines having the same phenotype; or
iv. including similar protein profiles; and
wherein the related bioactivity is preferably selected from bioprotection and biofertilizer activity; and
wherein the plant phenotype is preferably for drought tolerance or drought resistance.

70. The method according to claim 60, wherein prior to profiling endophyte strains from a microbiome, plant microbiome profiling is performed, said plant microbiome profiling including the steps of:
providing plant material from a first plant species and plant material from a related second plant species;
characterising the microbiome of the first and second plant species by analysing the plant material; and
assessing the microbiome of the first and second plant species to identify endophyte strains found in both the first and second plant species, or endophyte strains found in the second plant species but not in the first plant species.

71. A method for profiling a plant microbiome said method including the steps of:
providing plant material from a first plant species and material from a related second plant species, preferably wherein said first plant species is selected from perennial ryegrass (Lolium perenne), tall fescue (Festuca arundinaceae), corn (Zea mays), Glycine species including Glycine tomentella, Glycine tabacina, Glycine latifolia, Glycine hirticaulis, Glycine microphylla, Glycine clandestine and Glycine Max, wheat (Triticum aestivum) and barley (Hordeum vulgare), and preferably wherein the second plant species is a crop wild relative (CRW) of the first plant species;
characterising the microbiome of the first and second plant species by analysing the plant material; and
assessing the microbiome of the first and second plant species to identify endophyte strains found in both the first and second plant species or endophyte strains found in the second plant species but not in the first plant species.

72. The method according to claim 71, wherein providing the plant material from the first and second plant species includes the steps of:
sterilising the plant material; and
germinating the plant material,
wherein the germinated plant material provides seedlings for characterisation; and
wherein the plant material is preferably selected from seeds, leaves, stems, petioles, roots, buds, flowers or any combination thereof.

73. The method according to claim 71, wherein characterising the microbiome of the first and second plant species includes the steps of:
extracting nucleic acid from the plant material; and
analysing the extracted nucleic acid to profile the plant material microbiome.

74. The method according to claim 71, wherein assessing the microbiome of the first and second plant species includes statistical analysis to determine microbiome operational taxomic units (OTU) present within each plant species.

75. The method according to claim 74, wherein the microbiome OTU within the first plant species are compared to that of the second plant species to provides a means for determining microbiota which are either:
shared between the plant species, or
unique to the second plant species, relative to the first plant species.

76. The method according claim 74, wherein the identified microbiome OTU are selected from the group including Stenotrophomonas sp., Pseudomonas sp., Acinetobacter sp., Holomonas sp., Enterobactereaceae sp., Pantoea sp., Burkholderiaceae sp., Ralstonia sp., Massilia sp., Herbaspirillum sp., Delftia sp., Curvibacter sp., Aquabacterium sp., Sphingomonas sp., Novosphingobium sp., Bradyrhizobium sp., Ochrobactrum sp., Methylobacterium sp., Lactobacillus sp., Staphylococcus sp., Bacillus sp. and Curtobacterium sp.

77. A method of enhancing the bioactivity of a plant species, said method including:
identifying endophyte strains found in the first and second plant species according to the method of claim 71; and
transferring one or more endophyte strains from the second plant species to the first plant species to enhance bioactivity of the first plant species;
wherein the transferred endophyte enhances bioactivity of the first plant species; or
said method including identifying endophyte strains found in the second plant species but not in the first plant species, according to the method of claim 71, and
transferring one of said more endophyte strains from the second plant species to the first plant species,
wherein the transferred endophyte enhances bioactivity of the first plant species.

78. A substantially purified or isolated endophyte strain selected and/or isolated by the method according to claim 60, preferably wherein said endophyte is a strain of Xanthomonas sp., more preferably wherein the Xanthomonas sp. strain is GW as described herein and as deposited with The National Measurement Institute on 17 May 2019 with accession number V19/009902; preferably wherein said endophyte is a strain of Arthrobacter sp., more preferably wherein the Arthrobacter sp strain is D4-11 as described herein and as deposited with The National Measurement Institute on 9 Jul. 2019 with accession number V19/013680; preferably wherein said endophyte is a strain of Papiliotrema sp., more preferably wherein the Papiliotrema sp strain is selected from the group consisting of P2-Gland-NS-Runn creek IS-107-1, P1-Geland-NS-Mornington-IS-114-1, and P2-Geland-NS-Card Creek IS-34-1, as described herein and as deposited with The National Measurement Institute on 9 Jul. 2019 with accession numbers V19/013679, V19/013678 and V19/013677, respectively; preferably wherein said endophyte is a strain of Sphingomonas paucimobilis, more preferably wherein the Sphingomonas paucimobilis strain is Gtom-P2-19 as described herein and as deposited with The National Measurement Institute on 9 Jul. 2019 with accession number V19/013676; preferably wherein said endophyte is a strain of Argobacterium larrymoorei, more preferably wherein said Argobacterium larrymoorei strain is selected from Gcla-P1-10, and Gtab-P2-18, as described herein and as deposited with The National Measurement Institute on 9 Jul. 2019 with accession numbers V19/013675 and V19/013671, respectively; preferably wherein said endophyte is a strain of Pseudomonas oryzihabitans, more preferably wherein the Pseudomonas oryzihabitans strain is selected from Ghir-A-22-2, Gtab-P2-12 and Gtom-P2-14, as described herein and as deposited with The National Measurement Institute on 9 Jul. 2019 with accession numbers V19/013672, V19/013674 and V19/013673, respectively.

79. A plant, plant part or plant product with enhanced bioactivity produced by the method according to claim 60.
